(19) United States
(12) Patent Application Publication
(10) Pub. No.: US 2019/0247135 A1
(43) Pub. Date: Aug. 15, 2019

(54) MEDICAL DRAPERIES AND METHODS OF USE

(71) Applicant: Jeffrey E. McAlister, Phoenix, AZ (US)

(72) Inventor: Jeffrey E. McAlister, Phoenix, AZ (US)

(21) Appl. No.: 15/898,215

(22) Filed: Feb. 15, 2018

(51) Int. Cl. A61B 46/20

(52) U.S. Cl. CPC A61B 46/20

(57) ABSTRACT

Surgical draperies and methods of use are disclosed which provide a technique to prevent the edges of the surgical draperies from touching the floor. An elastic is included in a bottom portion of the surgical drape which provides for a snug connection to an operating table. Further elastic connecting bands or connecting devices such as hooks serve to further prevent the contact of the surgical drape with the floor.

### TECHNICAL FIELD

The subject disclosure relates to medical draperies. More particularly, the subject disclosure relates to medical draperies which are designed to connect to, enclose, and/or encompass surgical tables and beds.

### BACKGROUND

Surgical draperies (“drapes”) are commonly used to provide a sterile barrier between a surgically prepped area, which can be, for example, for operation on a limb, and a non-sterile area. This is typically accomplished with an elastic hole in the drape for the extremity. The material around the hole may be reinforced with a thicker, multilayered fabric.

Surgical drapes are often placed over a patient who is lying on an operating room table. Operating room tables generally have a table height range of 26″ to 45″ (660 to 1143 mm). A common drape utilized in the operating room has a width of 90″ (229 cm) which allows for less than optimal “over-coverage,” i.e., excessive drape on the floor when the table is at its minimum height.

Extremity surgeons will typically sit while operating to minimize risk of cervical and lumbar spine issues, and for general endurance and comfort. Depending on a number of factors, the extremity drape will typically touch, or even completely lay on, the floor. Very often the wheels of a surgeon's seat will roll over the drape and pull the drape (and operative limb), which can become problematic. Also, when using various operating room equipment, including, for example, C-arm X-rays, the drape may accidentally become unsterile by contact with the operating room equipment or personnel. Also, when using a C-arm X-ray system that requires a pedal, or arthroscopy kits utilizing a foot pedal, these items get “lost” or hidden underneath the drapes and decrease the efficiency of the surgical operation or break the concentration of the surgeon who has to readjust the drape to reach the foot pedal. A basic problem of having a universal extremity drape becomes more than an ease of use issue, but a sterility issue.

### SUMMARY OF THE SUBJECT DISCLOSURE

The present subject disclosure presents novel drapery designs and methods for use which are intended to overcome the common shortcomings of the conventional drapes. Furthermore, the present designs are intended to provide extremity “arm/wrist/hand/leg/ankle/foot” surgeons and operating staff a decreased risk of surgical site infection (SSI), ease of operating room equipment use, such as C-arm units, and less likelihood of “rolling over” the drape with a seat.

In one exemplary embodiment, the present subject matter is a surgical drapery. The surgical drapery includes a top horizontal portion having a width and a bottom edge; a bottom vertical portion having a width, a top edge a bottom edge, a left edge, and a right edge, and connected at its top edge to the bottom edge of the top horizontal portion, wherein the width of the top horizontal portion is larger than the width of the bottom vertical portion; an extended bottom portion extending beyond the left edge, the right edge, and the bottom edge the bottom vertical portion; and an elastic positioned at an outermost edge of the extended bottom portion.

In another exemplary embodiment, the present subject matter is a surgical drapery. The surgical drapery includes a top horizontal portion having a width and a bottom edge; a bottom vertical portion having a width, a top edge a bottom edge, a left edge, and a right edge, and connected at its top edge to the bottom edge of the top horizontal portion, wherein the width of the top horizontal portion is larger than the width of the bottom vertical portion; a window positioned on the bottom vertical portion; an extended bottom portion extending beyond the left edge, the right edge, and the bottom edge the bottom vertical portion; an elastic positioned at an outermost edge of the extended bottom portion corresponding to the left side portion, the right side portion, and the bottom portion of the vertical bottom portion extended bottom portion extending beyond the left edge, the right edge, and the bottom edge the bottom vertical portion.

In yet another exemplary embodiment, the present subject matter is a method of applying a surgical drapery to an operating table. The method includes proving a surgical drapery which includes a top horizontal portion having a width and a bottom edge; a bottom vertical portion having a width, a top edge a bottom edge, a left edge, and a right edge, and connected at its top edge to the bottom edge of the top horizontal portion, wherein the width of the top horizontal portion is larger than the width of the bottom vertical portion; an extended bottom portion extending beyond the left edge, the right edge, and the bottom edge the bottom vertical portion; and an elastic positioned at an outermost edge of the extended bottom portion. Further, the method includes placing the surgical drapery over the operating table such that the elastic is positioned on the underside of the operating table to prevent any edges of the bottom vertical portion from extending beyond any edges of the operating table or making contact with a floor.

### BRIEF DESCRIPTION OF THE DRAWINGS

Various exemplary embodiments of this disclosure will be described in detail, wherein like reference numerals refer to identical or similar components or steps, with reference to the following figures, wherein:

FIG. 1 illustrates a patient positioned on an operating table during extremity surgery.

FIG. 2 illustrates a top view of a surgical drapery, according to an exemplary embodiment of the present subject disclosure.

FIG. 3 illustrates a top view of another surgical drapery, according to another exemplary embodiment of the present subject disclosure.

FIG. 4 illustrates a top view of another surgical drapery, according to another exemplary embodiment of the present subject disclosure.

FIG. 5 illustrates a top view of another surgical drapery, according to another exemplary embodiment of the present subject disclosure.

### DETAILED DESCRIPTION

Particular embodiments of the present subject disclosure will now be described in greater detail with reference to the figures.

The subject disclosure is described with reference to the drawings, wherein like reference numerals are used to refer to like elements throughout. In the following description, for purposes of explanation, numerous specific details are set forth in order to provide a thorough understanding of the present disclosure. It may be evident, however, that the present disclosure may be practiced without these specific details.

As employed in this specification and annexed drawings, the term “or” is intended to mean an inclusive “or” rather than an exclusive “or.” Moreover, articles “a” and “an” as used in the subject specification and annexed drawings should generally be construed to mean “one or more” unless specified otherwise or clear from context to be directed to a singular form.

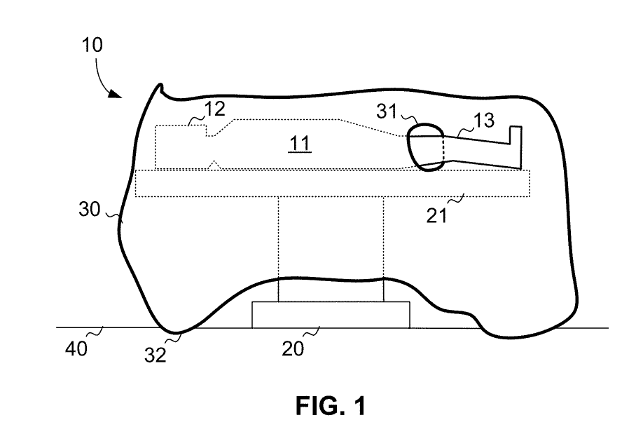

FIG. 1 shows a typical configuration 10 for a patient 11 positioned for surgery, particularly for extremity surgery. The patient 11 is positioned supine (face up) atop the substantially horizontal platform 21 of an operating table 20. Alternatively, the patient 11 may be positioned prone (face down) or on either side, as a surgeon would deem appropriate for the type of surgery to be performed. The platform 21 may be positioned substantially horizontally as shown such that it is parallel to the ground or, alternatively, at an angle such that the either the head 12 or leg 13 portion of the patient is higher than the leg 13 or head 12 portion, respectively.

In many types of surgery, it is conventional practice to use a drape 30 to substantially cover the patient 11 during the operation to prevent debris or other material from the operation to come in contact with the patient 11, and to also isolate, and maintain sterile, the portion of the patient which is undergoing the procedure. The drape 30 may also serve to keep the patient 11 warm and comfortable during the procedure.

As shown in FIG. 1, the drape 30 has an isolated open window 31 which allows a portion of the body of the patient 11 to come through from under the drape 30. In the example shown, the leg 13 is brought through the opening 31 from under the drape 30 to outside the drape. This way, the surgeon can isolate the leg 13 to be operated on and create a sterile field for just the subject leg 13. While a conventional drape 30 may adequately cover the patient 11 and isolate the portion of the patient's body, e.g., leg 13, for a surgical procedure, the outer edges 32 of the drape 30 tend to overflow and make contact with the non-sterile floor 40, thereby compromising the general sterility of the drape 30.

Further, as previously stated, because the edges 32 of the drape 30 are overflowing, this tends to prevent the use of various operating room equipment, like C-arms, etc., without the operating room equipment making contact with the drape 30 and compromising its sterility. The surgeon or other operating room personnel may also accidentally step on the edges 32 or run it over with the wheels of a chair or other equipment, such as intravascular (IV) pole, etc. By stepping or running over the edge 32 of the drape 30, not only will this compromise the general sterility of the drape 30, but will also result in a possible pulling of the drape 30 in the direction where the drape edge 32 was stepped on or run over. This would result in the operating window 31 moving or shifting toward the direction of the unsterile contact which could cause damage to the surgical site because the window 31 edge could come into direct contact with an open surgical site and pull or tear that site. Thus, any stepping on or rolling over the edge 32 of the drape 30 during an operation can cause both sterility issues and physical contact damage issues with the surgical site.

The subject disclosure addresses these shortcomings and provides many further advantages to the operating room surgeon and personnel to decrease the risks from operations using conventional medical drapes. This present subject disclosure, through its various exemplary embodiments described below and shown in the accompanying drawings, is intended to provide extremity arm/wrist/hand/leg/ankle/foot surgeons and operating staff a decreased risk of surgical site infection (SSI), ease of operating room C-arm use, and less rolling over the drape. This may be solved by adding an elastic or rubber band-type fabric around the portion of the drape covering the subject limb only. This will pull all the remaining material underneath the operating table and allow for a reduction of the above-mentioned risks.

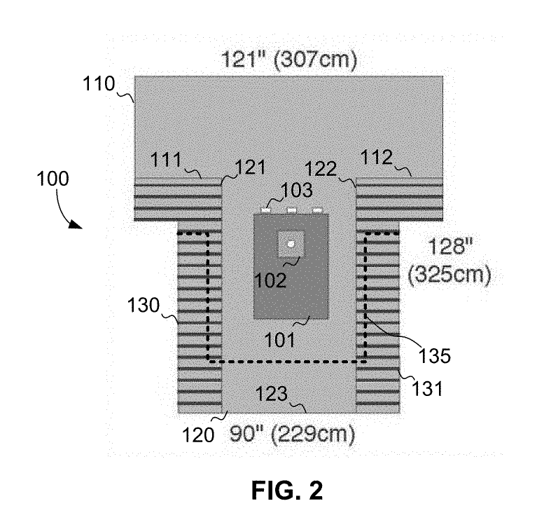

FIG. 2 illustrates one exemplary embodiment of the present subject disclosure. This exemplary drape 100 has a general T-shaped configuration with a top horizontal portion 110 and a bottom vertical portion 120. Material extension portions 130/131 are extend from and beyond the left side edge 121 and right side edge 122 of the vertical portion 120, respectfully. The top portion 110 has a wider width than the width of the bottom portion 120 and the combined bottom sides of the left 130 and right extended side 131 portions. The left side material extension 130 is connected to the top horizontal portion 110 at border 111 on the left side and border 112 at the right side. Thus, the left side material extension 130 is attached directly to both the top horizontal portion 110 at border 111, and the bottom vertical portion 120 and border 121. Similarly, the right side material extension 131 is attached directly to both the top horizontal portion 110 at border 112, and the bottom vertical portion 120 at border 122.

A window 101 is positioned within the bottom vertical portion 120 and has a hand engaging portion 102 to pull open the window and connectors 103 (e.g., hinges or the like) which connect the openable window 101 upward to access the subject body portion which is the intended target of the surgical procedure. The window 101 is shown spanning the top horizontal portion 110 and the bottom vertical portion 120, but it may be positioned anywhere it would be beneficial for a particular surgical operation.

Left side portion 130 and right side portions 131 may be constructed of a type of material which can more easily conform to the shape of a horizontal bed platform 21 (as shown in FIG. 1). The material of left 130 and ride side 131 portions may have elastic qualities and be fenestrated to help attach the bottom vertical portion 120 to the horizontal bed platform 21. A fenestrated left 130 and right 131 side portions would allow for better pulling and overlying the sides of the horizontal bed platform 21.

An elastic material or band 135 is connected from an outermost edge of the left side portion 130 to an outermost edge of the right side portion 131. This band 135 can extend directly underneath the bottom portion of the horizontal bed platform 21 and pull the left 130 and right 131 side portions tight against the upper surface of the horizontal bed platform 21. The band 135 may be continuous from one edge of connection to the left side portion 130 to the other edge of connection to the right side portion 131. Alternatively, the band 135 may be two or more parts which are connectable together through standard connection mechanisms known and appreciated by one having ordinary skill in the art (VELCRO, hook and loop, belt buckle, complementary hooks, etc.).

The use of the band 135 would ensure that the bottom vertical portion 120 of the surgical drape 100 would fit snugly against the horizontal bed platform 21. Thus there would be no edges 32 (as shown in FIG. 1) on either side of the drape 100 which could be contaminated by contact with the floor 40. Further, any surgical equipment (such as a C-arm) may be readily used to perform functions as needed on the patient while minimizing the possibility of contact. Finally, because the edges of the left side portion 130 and right side portion 131 of the drape 100 are securely positioned against the underside of the horizontal bed platform 21, there is a much decreased chance of the edge of the drape 100 being stepped on or rolled over during operation, thereby causing contamination or moving the subject limb during the delicate operation.

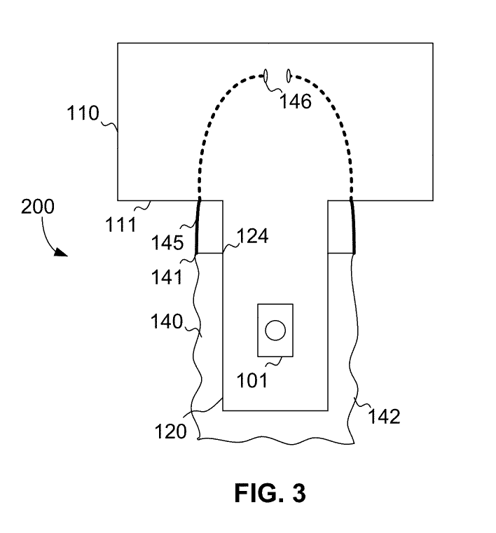

FIG. 3 shows another exemplary embodiment of a surgical drape 200 according to the present subject disclosure. This embodiment 200 has the same general overall shape as that shown in FIG. 2, including a horizontal top portion 110, a vertical bottom portion 120 and openable access window 101. However, this embodiment 200 has certain unique characteristic and features. For example, there is a continuous extended bottom portion 140 which extends around most of the outer edge of the bottom vertical portion 120. The extended bottom portion 140 extends vertically to a top side line 141, which is connected to a side 124 of the vertical bottom portion 120, and does not contact the horizontal upper portion 110, as did the embodiment shown in FIG. 2.

The material used for extended bottom portion 140 may be elastic, fenestrated or otherwise the same or softer than the material used for the rest of the drape 200.

An elastic 142 may be positioned on the very outer edge of the extended bottom portion 140, which includes the three sides of the left, bottom, and right sides. Thus, the elastic band 142 extends on three of the four sides of the extended bottom portion 140.

Further, another elastic band 145 extends from the upper side 141 of the extended bottom portion 140 and is connected on both the left and right sides of the extended bottom portion 140. The band 145 may be similar to the band 135 shown and described in FIG. 2. The band 145 shown in FIG. 3 has two component portions which are attached to the left and right sides of the upper portion of the extended bottom portion 140, and has a complementary connecting mechanism 146 to attach the two (or more) component portions together. The band 145 and attachment mechanism 146 provide versatility and flexibility in attaching the vertical bottom portion 120 to the top of the horizontal bed platform 21 (shown in FIG. 1) and securing it through various ways and positions, depending on the underside configuration of the bed platform 21.

The positioning of the band 145 at an upper end of the extended bottom portion 140, which is positioned below the bottom edge 111 of the upper horizontal portion 110 allows for tighter fit of the bottom vertical portion 120 around the bed platform. The position of the window 101 is located generally lower on the bottom vertical portion 120. This configuration would be helpful in operations where the furthest extremities are to be operated on (e.g., ankles, feet, toes) because the elastic portion 142 would be concentrated on the lowest ends of the drape 200.

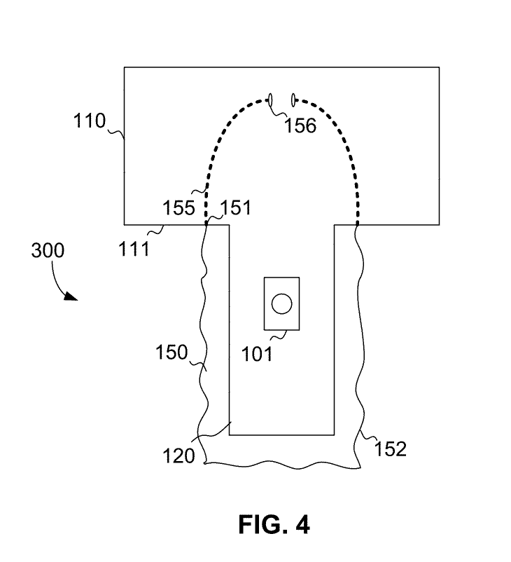

FIG. 4 shows an alternative version 300 of the embodiment 200 shown in FIG. 3. The drape 400 is substantially similar to the embodiment 200 shown and described in FIG. 3 in that there is a horizontal top portion 110, a vertical bottom portion 120, and window 101. However, the extended bottom portion 150 extends the entire outer edge of vertical bottom portion 120 such that the top edge of the extended bottom portion 150 comes into contact with and connects to the horizontal top portion 110 at its bottom edge 111.

An elastic band 152 surrounds the entire length of the three sides of the extended bottom portion 150. A further elastic band 155, similar to bands 135 and 145, is connected to a point 151 on the bottom edge 111 of the connection between the horizontal top portion 110 and the extended bottom portion 150. The band 155 also has connectors 156 similar to the ones 146 described for connectors 145 of FIG. 3. The particular configuration shown in FIG. 4 has a window portion 101 that is positioned generally higher on the vertical bottom portion 120 and may be more helpful where the operable site is higher than the further extremities (e.g., ankles or calves, etc.) because the elastic portion 152 would be extended across the entire length of the vertical bottom portion 120 of the drape 300. Although specific positions of the window 101 are shown in the various described embodiments, the window 101 may be placed anywhere on the drape as needed in positions which would be most helpful to isolate the intended body portion which is the subject of an operation. The particular position of the window 101 shown in any particular embodiment is not limited to that exact position, but may be changed as appreciated by one having ordinary skill in the art.

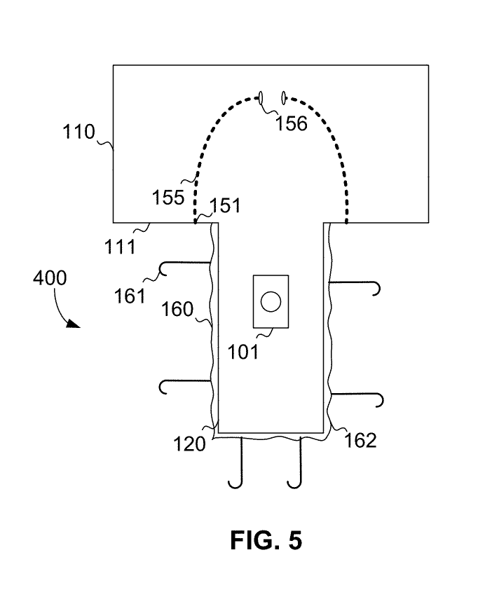

FIG. 5 shows another exemplary embodiment of a drape 400 according to the present subject disclosure. The drape 400 is substantially similar the drape 300 shown and described in FIG. 4 in that there is a horizontal top portion 110, a vertical bottom portion 120, window 101, and a securing elastic band 155 connected to a point 151 on the bottom edge 111 of the top horizontal portion 110. Further, there is an extended bottom portion 160 which spans the entire outer edge of the vertical bottom portion 120, and contains an elastic 162 to help secure the bottom vertical portion 120 to the top of a horizontal bed platform 21 (shown in FIG. 1).

However, the extended outer edge 160 may have less material or a smaller width than the extended bottoms portions 140 and 150 shown in the prior exemplary embodiments of FIGS. 3 and 4, respectively. A connection device 161 or a series of connection devices 161 are positioned to extend beyond the outer edges of the extended bottom portion 160. These connection devices may be any device that is able to hook onto the various bottom configurations of a surgical bed 20, and may include hooks, clips, snaps, or the like.

The exemplary embodiment 400 shown in FIG. 5 shows the connection devices 161 as hooks for sake of complicity, but they are not limited to hooks and may be other devices to secure the extended bottom portion 160 of the drape 400, as appreciated by one having ordinary skill in the art. The connection devices 161 may be positioned on one, two, or three sides (as shown) of the extended bottom portion 161 and may be adapted to connect to each other or to any rod, tube, ledge, hole, corner or other connectable part of the horizontal bed platform 21 or the operating table 20 (shown in FIG. 1).

What has been described above includes examples that provide advantages of the subject disclosure. It is, of course, not possible to describe every conceivable combination of components or methodologies for purposes of describing the subject disclosure, but one of ordinary skill in the art may recognize that many further combinations and permutations of the claimed subject matter are possible. Furthermore, to the extent that the terms “includes,” “has,” “possesses,” and the like are used in the detailed description, claims, appendices and drawings such terms are intended to be inclusive in a manner similar to the term “comprising” as “comprising” is interpreted when employed as a transitional word in a claim.

The illustrations and examples provided herein are for explanatory purposes and are not intended to limit the scope of the appended claims. It will be recognized by those skilled in the art that changes or modifications may be made to the above described embodiment without departing from the broad inventive concepts of the subject disclosure. It is understood therefore that the subject disclosure is not limited to the particular embodiment which is described, but is intended to cover all modifications and changes within the scope and spirit of the subject disclosure.

What is claimed is:

1. A surgical drapery, comprising:
a top horizontal portion having a width and a bottom edge;
a bottom vertical portion having a width, a top edge a bottom edge, a left edge, and a right edge, and connected at its top edge to the bottom edge of the top horizontal portion, wherein the width of the top horizontal portion is larger than the width of the bottom vertical portion;
an extended bottom portion extending beyond the left edge, the right edge, and the bottom edge the bottom vertical portion; and
an elastic positioned at an outermost edge of the extended bottom portion.

2. The surgical drapery of claim 1, wherein the elastic in the extended bottom portion corresponds to the left side portion, the right side portion, and the bottom portion of the vertical bottom portion.

3. The surgical drapery of claim 1, further comprising a flexible band connected to the bottom edge of the top horizontal portion.

4. The surgical drapery of claim 3, wherein the flexible band comprises a single structure that is connected on both sides to the bottom edge of the top horizontal portion.

5. The surgical drapery of claim 3, wherein the flexible band comprises multiple parts that are connected on both sides to the bottom edge of the top horizontal portion.

6. The surgical drapery of claim 5, wherein the multiple parts of the flexible band connect together.

7. The surgical drapery of claim 1, further comprising a flexible band connected to a top edge of the extended bottom portion.

8. The surgical drapery of claim 7, wherein the flexible band comprises a single structure that is connected on both sides to the top edge of the extended bottom portion.

9. The surgical drapery of claim 7, wherein the flexible band comprises multiple parts that are connected on both sides to the top edge of the extended bottom portion.

10. The surgical drapery of claim 9, wherein the multiple parts of the flexible band connect together.

11. The surgical drapery of claim 1, wherein a top edge of the extended bottom portion is connected to the bottom edge of the top horizontal portion.

12. The surgical drapery of claim 1, wherein a top edge of the extended bottom portion is connected to the left side and the right side of the bottom vertical portion.

13. The surgical drapery of claim 1, further comprising a window positioned on the bottom vertical portion.

14. The surgical drapery of claim 1, further comprising connecting devices positioned on an outermost edge of the extended bottom portion.

15. The surgical drapery of claim 14, wherein the connecting devices are positioned on a left side, a right side, and a bottom side of the extended bottom portion.

16. The surgical drapery of claim 15, wherein the connecting devices comprise hooks.

17. A surgical drapery, comprising:
a top horizontal portion having a width and a bottom edge;
a bottom vertical portion having a width, a top edge a bottom edge, a left edge, and a right edge, and connected at its top edge to the bottom edge of the top horizontal portion, wherein the width of the top horizontal portion is larger than the width of the bottom vertical portion;
a window positioned on the bottom vertical portion;
an extended bottom portion extending beyond the left edge, the right edge, and the bottom edge the bottom vertical portion;
an elastic positioned at an outermost edge of the extended bottom portion corresponding to the left side portion, the right side portion, and the bottom portion of the vertical bottom portion extended bottom portion extending beyond the left edge, the right edge, and the bottom edge the bottom vertical portion.

18. The surgical draper of claim 17, further comprising a flexible band connected to the bottom edge of the top horizontal portion.

19. The surgical drapery of claim 1, further comprising a flexible band connected to a top edge of the extended bottom portion.

20. A method of applying a surgical drapery to an operating table, comprising:
proving a surgical drapery comprising:
a top horizontal portion having a width and a bottom edge;
a bottom vertical portion having a width, a top edge a bottom edge, a left edge, and a right edge, and connected at its top edge to the bottom edge of the top horizontal portion, wherein the width of the top horizontal portion is larger than the width of the bottom vertical portion;
an extended bottom portion extending beyond the left edge, the right edge, and the bottom edge the bottom vertical portion; and
an elastic positioned at an outermost edge of the extended bottom portion; and
placing the surgical drapery over the operating table such that the elastic is positioned on the underside of the operating table to prevent any edges of the bottom vertical portion from extending beyond any edges of the operating table or making contact with a floor.
